(19) United States
(12) Patent Application Publication
(10) Pub. No.: US 2009/0076565 A1
(43) Pub. Date: Mar. 19, 2009

(54) METHODS FOR TREATING URINARY AND FECAL INCONTINENCE

(75) Inventor: Earl A. Surwit, Tucson, AZ (US)

Correspondence Address: Pabst Patent Group LLP, 1545 PEACHTREE STREET NE, SUITE 320, ATLANTA, GA, 30309, US

(73) Assignee: State of Incorporation

(21) Appl. No.: 11/857,872

(22) Filed: Sep. 19, 2007

(51) Int. Cl. A61N 1/05; A61N 1/36

(52) U.S. Cl. 607/41; 607/144

(57) ABSTRACT

Non-surgical methods for treating pelvic floor muscular dysfunctional disorders are provided. The method combines pelvic floor muscle training (PFMT), biofeedback and pelvic floor exercises, pudental and hypogastric nerve neuromodulation (electrical stimulation), and tibial nerve neuromodulation (PTNS).

### FIELD OF THE INVENTION

Methods and systems for treating or inhibiting conditions related to weakening of the pelvic floor, including but not limited to urinary and fecal incontinence as well as other pelvic floor dysfunctional diseases, such as overactive bladder (“OAB”), pelvic organ prolapse (“POP”), pelvic pain, severe constipation, and fecal impaction, using electrical stimulation and electrical neuromodulation in combination with exercise.

### BACKGROUND OF THE INVENTION

Urinary incontinence in the United States is an extremely common problem with a prevalence of 25% of women, age 30-60 years, 38% of women age 60-70 years and 56% of women in chronic care facilities having incontinence. Stress incontinence is alleged to be the most common form of urinary incontinence. The true incidence of urge incontinence that needs to be treated, both the pure urge and mixed incontinence, accounts for 56% of women who are incontinent. Add the additional 33 million women with overactive bladders, and you have a large percentage of the American population.

Only 20%-30% of patients with urinary incontinence seek help. The primary reason for not seeking professional help is their fear of surgery. The impact on quality of life can be substantial with the most severe of restriction leading to a housebound status. Incontinence also has a significant adverse impact on sexual functions if leakage occurs during sexual intercourse. Vulvar and chronic dermatitis are also common side effects of urinary incontinence, secondary to frequent contact with wet security pads and diapers. Quality of life surveys for patients with urinary incontinence have shown the same low scores as patients with severe coronary artery disease, severe diabetes, depression, and significant rheumatoid arthritis. Urinary incontinence increases the risk of bone fractures by 45% and increases the risk of hospitalization, 30% in women and 50% in men. Urinary incontinence is the second most common cause for admission to nursing homes.

The pathophysiology of neurological pathways and muscarinic dysfunction is important in order to understand the etiology of urinary incontinence. Urinary incontinence to a large extent stems from a weak pelvic floor musculature. The pelvic floor muscles provide numerous functions related to the appropriate functioning of the lower urinary tract. Contraction of these muscles will quiet the bladder down through reflex neurological pathways, back and forth to the neurological sacral complex of nerves. These muscles prevent pelvic organ prolapse, which frequently leads to overflow incontinence in women. A strong pelvic floor musculature will prevent genuine stress incontinence by supporting the bladder base and urethra. The pelvic floor musculature and bladder receive the majority of their stimulation and or inhibition through the pudental nerve. The pathway to and or from the pudental nerve goes to the spine predominantly at S3, then S2, and a small component to S4.

Normal bladder function can be seen as having both a filling and emptying phase. During the filling phase, the sympathetic neurological system (including the sacral neurological network and Onuf's nucleus in the lower lumbar spine) allows the bladder to fill while maintaining the urethral pressure. This makes the bladder a unique organ in the body, as the pressure inside the bladder does not rise with volume. This is accomplished through a complex feedback mechanism in the bladder wall. Any increase in abdominal pressure will be transmitted directly to the bladder, raising the bladder pressure as well.

During the emptying phase, the bladder (detrusor) muscle contracts the bladder wall through the parasympathetic neurological pathways, the sacral complex and parasympathetic nucleus. The urethra relaxes, the pelvic floor relaxes and the patient voids. Therefore, incontinence can be seen simply as when the bladder pressure is greater than the urethral pressure at that moment of urinary loss.

The anatomy of these neurological pathways for micturition is important. The pelvic floor and bladder receive the majority of their stimulation or inhibition from the pudental nerve, shown in FIG. 1. The pathway from the pudental nerve to the spine goes predominantly to S3 and then the rest to S2, partially to S4. There is a complex reflex voiding circuitry involving large Beta fibers (afferent and efferent) and small c-fibers which are usually involved in afferent reflex arcs. However, if neuromodulation of the sacrum continues long enough, these c-fibers will take over as efferent pathways.

A review of the non-surgical treatment options for incontinence is found in, “Pelvic Floor Muscle Training and Adjunctive Therapies for the Treatment of Stress Urinary Incontinence in Women: A Systemic Review”, Neumann P, BMC Women's Health. 6:1 11-35 (2006). Four studies using vaginal electromyography (EMG) and biofeedback as a clinic treatment showed cure rates from 25-80% [Hay-Smith, The Cochran Database of Systematic Reviews, (1):1-115 (2001); Pages, Am J. Phys Med Rehabil, 80 (7): 494-502 (2001); Morkved, Obstetrics & Gynecology. 100(4): 730-739 (2002); Cammu, Eur J Obstet Gynecol Reprod Biol, 77(1):89-93 (1998); Berghmans, et al., 1998; Glavind, Int Urogynecol JPelvic Floor Dysfurnct, 7:339-343 (1996) with positive and statistically significant outcomes. Regarding the use of EMG and biofeedback, one study found no difference in outcomes with the addition of the biofeedback (BF) Wong, Physiotherapy, 87(12): 644-648 (2001) and another showed that there was a difference, Johnson, Nursing Research, 50(1):33-41(2001). There were no reports of the occurrence of adverse effects. The authors concluded in the substance of the paper “when considering all the studies on PFMT/BF a total of 25/29 (86%) demonstrated statistically significantly improvement and, while only 4 outcomes failed to show significant change after treatment, all of these occurred in two studies with treatment times of four and six weeks respectively (Inadequate treatment). The poor results seen in only 14% of all of the 24 studies reviewed may have been due to measurement error, as pad-tests without demonstrative reliability were used and because of the short duration of training, which may have been insufficient to effect physiological changes. This review concludes, “Thus in summary, there is strong evidence from a number of randomized control trials that pelvic floor medical treatment with vaginal EMG or pressure studies, that biofeedback is effective for the treatment of stress urinary incontinence, but it may be no more effective that PFMT alone.” There was a declining success over time reported corresponding with a decline in the PFMT exercise compliance. There were well validated studies on pelvic floor muscle training utilizing perineometry to measure the increase in strength of the pelvic floor muscles. [Dumoulin, Physical Therapy, 75(12):1075-1081 (1995); Lamers, Int Urogynecol J, 18:301-307 (2007); Cammu, Am J Ostet Gynecol 191(4):1152-1157 (2004)] that showed increases in strength. The study concluded that “There is strong evidence from a number of high quality randomized control trials that using a specific strength training protocol increases pelvic floor muscle strength with measurable changes between four weeks and six months, and leads to excellent cure rates for the patients”. Also, in accordance with physiological principles, evidence from this review confirmed that longer training times produced greater gains in strength.

Central sacral nerve stimulation was introduced into the United States for urge incontinence in 1995 [Bosch J Urol 154:504-507 (1995)]. It requires a temporary test stimulation in the hospital to see if the patient is going to respond to the more permanent implant. This test is done in the operating room with anesthesia and fluoroscopy, usually requiring some tissue dissection, otherwise, the test wire will migrate. This minor test can have complications. The permanent implant, which is a second hospital based procedure, has complications [Dijkema, Fur Urol. 24:72-76 (1993)]. These are pain at the neurostimulator implant site (15%), new pain (9%), lead migration (8%), and transient electric shock (6%), pain at the lead site (5%), technical problems (3%), and 6 other minor problems with a 1% or less incidence. The best results for sacral implants in urge incontinence are reported by Eljilali, Urology 65(6):1114-1117 (2005). Their definition of success was dry or a 50% improvement in symptoms. Utilizing this definition, they reported a 47% success rate. Unfortunately, there is also a significant revision rate, from 12% to 33%, A number of studies has reported that continuous neuromodulation, as given with central sacral implants will not worl, especially over time.

A percutaneous tibial nerve (PTNS) approach for sacral stimulation was first introduced in the United States in 1997 by Stoller, et al, 1997. An acupuncture point (Spleen 6-bladder and kidneys) is utilized for the placement of the acupuncture needle to neuromodulate the tibial nerve. Kokli states that this “office-based convenience makes it (PTNS) an ideal initial neuromodulation procedure for the treatment of OAB, voiding dysfunction, and pelvic pain” [Kokli Clin Obstet Gynecol. 45(1):218-232 (2002). PTNS consistently leads to a 40% dry rate in urinary urge incontinence, with a 70% overall cure/improvement rate. PTNS is an outpatient 30 minute painless procedure that is easily mastered by both physicians and nurses. The early work with PINS usually employed a total of 12 weekly treatments. Additional maintenance treatments were also employed in some of these early studies.

The standard of care in the world today for stress urinary incontinence is usually a surgical procedure. This encompasses a sub-urethral sling either at the mid-urethra such as a trans-vaginal tape (TVT) or a sling at the bladder neck such as a spark procedure utilizing various materials for the sling. Alternative approaches such as transobturator stings are also being utilized. The Burch supra-pubic suspension also still has a place in the treatment of stress incontinence. A review of surgery utilizing an anterior repair of a cystocele (“bladder tuck”), or an anterior repair along with a posterior repair of a rectocele (“pocket back there”), plus or minus a vaginal hysterectomy (Vag Hyst A & P repair) reveals 11 studies in 957 patients with a cure rate of 65% [Cardoza Textbook of Female Urology and Urogynaecology ISIS Medical Media. 2001]. The procedure only lasts for an average of 5 years for the patient and is being done on patients with mixed incontinence, that is stress and urge incontinence with an overactive bladder. This will frequently lead to a worsening of the urge component of the incontinence. The American Urogynecologic Society reports an 83% cure rate for slings and a 79% cure rate in a multivariate analysis of 28 studies and 4,394 patients for retropubic cystourethropexies. The problem with the majority of these studies is that they use the International Continence Society's definition of cure, which is a greater than an 80% improvement for the patient. Women want to be dry and not just have improvement. The patients want to be cured; dry, no more pads, and most importantly no more fear of incontinence. This fear can only be dismissed, if the patient is truly cured for the long term. The surgical procedure for urge urinary incontinence is central sacral implants, as noted above.

The treatment of urinary incontinence should always include; 1) extensive dietary counseling, that is the avoidance of caffeine and acidic foods, 2) behavioral modification and emotional support by the nursing staff and physician, 3) vaginal estrogen cream and 4) long-term follow-up with vaginal manometry to measure the pelvic floor strength to be reassured that the patient continues to do their pelvic floor muscle exercise. The diet must be constantly reinforced by the nurses and the patient must completely eliminate coffee, decaf coffee, caffeinated tea and chocolate along with any medications that contain caffeine. Acidic foods will also irritate the bladder and the patient has the choice to eliminate them prior to treatment or they can test them to see if they are irritants.

Vaginal estrogen therapy is critical for the treatment of urinary incontinence and prolapse. The bladder is an estrogen dependent organ which was demonstrated by cytological studies in the 1940s where PAP smears were taken from menstruating women in the vagina and bladder and showed the identical hormonal changes in both the bladder and the vagina. Pragmatically, the vaginal probe treatment with pelvic floor rehabilitation will be uncomfortable in an atrophic vagina. Kegel's exercises are relatively worthless as normally taught. The patient may well be exercising the thighs, buttocks and abdominal wall muscles instead of the pelvic floor muscles. There is also no physician follow-up and most patients will quit doing the exercises on their own.

Electrical stimulation at 10-20 Hz [Cook, Instrrumentation Clinical Electrotherapy Second Edition: 2(4-19) (1996); Kralj Functional Electrical Stimulation, W.B. Saunders Company, Edition 7 48:508-509 (1974)] results in neuromodulation of the hypogastric nerve through the pudental nerve and activates the sympathetic vesicular-inhibitory neurons, Neuromodulation of the pudental nerve also will lead to afference to the spinal reflexes which inhibit the parasympathetic vesicle motor neurons. [Jarvis, BJOG: An international journal of Obstetrics and Gynaecology 111(10)1031-1035 (2004)] Electrical stimulation in the urethra is also a positive outcome up to 35 mA. [Richards, Int Urogynecol J. 2006; 17:672-678 (2006)] It will increase the maximal urethral pressure (MUP) and will increase the maximal urethral closure pressure (MUCP). It will cause reflex stimulation of the pelvic floor muscles improving pressure transmission to the urethra during stressful activity. This is important in both stress incontinence and intrinsic sphincter deficiency (ISD). Electrical stimulation is also sensory feedback to the patients of which muscles they need to exercise.

The medical treatment of urge incontinence across the world has traditionally been the use of such pharmaceutical agents such as antispasmodics, anticholinergics and imipramine. Side effects such as dry mouth and constipation are frequent. These pharmacologic interventions will improve incontinence for a large number of patients, but are not curative, and also not tolerated for many in the long term (Kelleher, C.J. et al. Br J Obstet Gynaecol.1997;104:993-998), They are, furthermore, potentially dangerous in older patients whose blood brain barrier has become less effective and hence more of the drug gets into the central nervous system (Mulsant, B.H. et al., Arch Gen Psychiatry.2003;60:198-203) with subsequent increased likelihood of side effects. The compliance rate at six months remains at 18-30%, [Smither, BMC Urology 7:2:doi:10.1186/1471-2490-7-2 (2007), secondary to efficacy, cost and side effects. The CNS side effects in older patients prevent the utilization of anticholinergics where the blood-brain barrier has begun to breakdown.

Thus, it is an object of the invention to provide improved non-surgical methods for the treatment of pelvic-floor related disorders.

It is an object to provide non-surgical methods for treating both urinary and fecal incontinence.

It is an object to provide non-surgical methods for treating pelvic organ prolapse.

It is another object to provide non-surgical methods for treating pelvic pain.

### SUMMARY OF THE INVENTION

Electrical stimulation (pudental and hypogastric nerve neuromodulation) in combination with tibial nerve neuromodulation accompanied by training and rigorous compliance with pelvic floor exercises in combination with biofeedback to insure the exercises are done appropriately is an effective method for treating pelvic-floor dysfunctional diseases, in particular urinary and fecal incontinence, as well as overactive bladders, pelvic organ prolapse, pelvic pain, severe constipation, and fecal impaction. The treatment is typically administered to woman (trans-vaginally) but can also be used to treat men (trans-anally, using the same corresponding muscles and nerve pathways). In the preferred embodiment, the patient is conducting pelvic floor exercises on a daily basis. Neurofeedback can be used to insure these are being properly performed. This can be done by applying electrical stimulation until there is sustained contraction of the pelvic floor muscles, for example, at 50 to 150 Hz, preferably 100 Hz, or a tingling at 5-20 Hz, preferably 10 Hz, Neurostimulation is applied to the pudental and hypogastric nerves using a vaginal or rectal probe to apply electrical stimulation. Neurostimulation is also applied to the tibial nerve using percutaneous (such as an acupuncture needle, from 0.1 to 15 mA, to stimulate the tibial nerve, most preferably from 0.25 mA up to 9 mA at 9 Hz). Transcutaneous stimulation of the tibial nerve with a Microcurrent pad stimulator, microcurrent probe stimulator, Russian device micro-needle patch, skin electrodes, or superficial transcutaneous probes can also be used to stimulate the tibial nerve between 0.1 mA to 50 mA, most preferably between 1 mA and 40 mA.

### BRIEF DESCRIPTION OF THE DRAWINGS

FIG. 1 is a diagram of the pelvic and sacral neurological complexes and the pudental nerve.

FIGS. 2A-2D are diagrams of the entire female pelvic musculature.

FIG. 3 is a diagram of the pudental nerve (marked by dot) demonstrating how the pudental nerve continues down the foot, neuromodulates the sacral neurological complex of nerves; and goes up to Onuf's nucleus in the lumbar spine.

### DETAILED DESCRIPTION OF THE INVENTION

One of the main underlying pathologies contributing to disorders of the pelvic floor such as urinary urge incontinence is a weak pelvic floor. It has been discovered that the combination of (1) traditional pelvic floor rehabilitation using biofeedback and (2) neuromodulation of the pudental and hypogastric nerves coupled with (3) neuromodulation of the tibial nerve is an effective method for strengthening the pelvic floor of an individual. Rehabilitating the pelvic floor using this combination of treatments restores the reflex arc, so that contraction of the pelvic floor muscles inhibits the detrusor muscle by a sacral reflex, quieting the bladder. Strengthening the pelvic floor is an important element in achieving a successful treatment with lasting duration for pelvic floor related disorders.

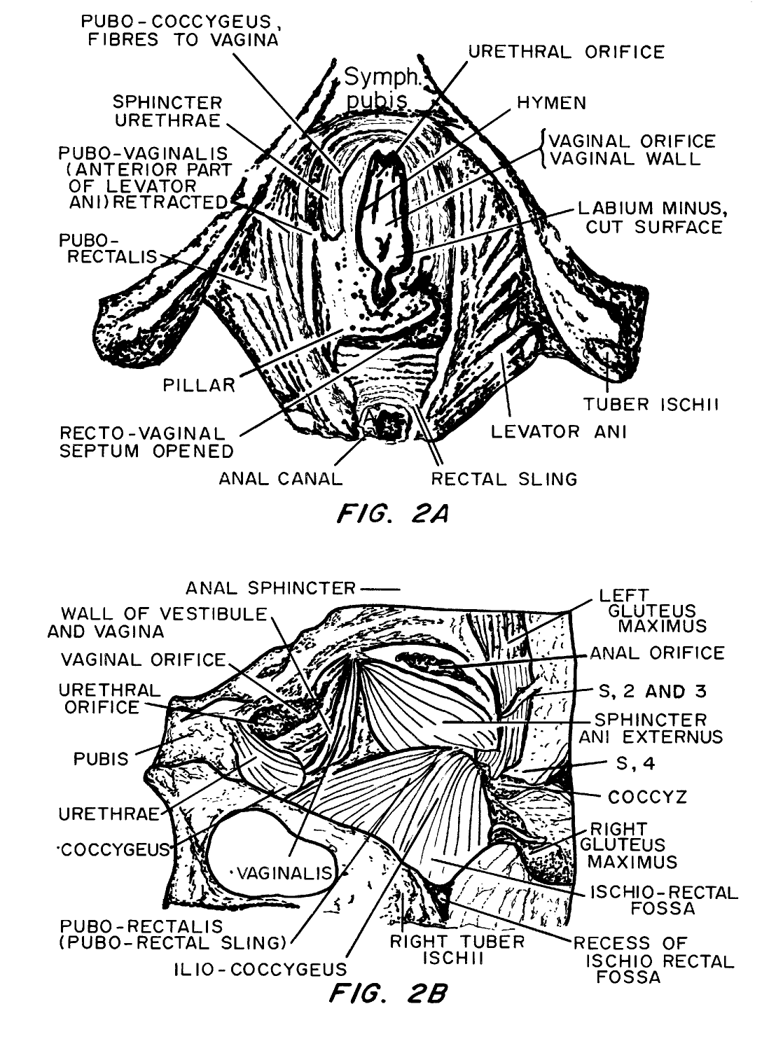

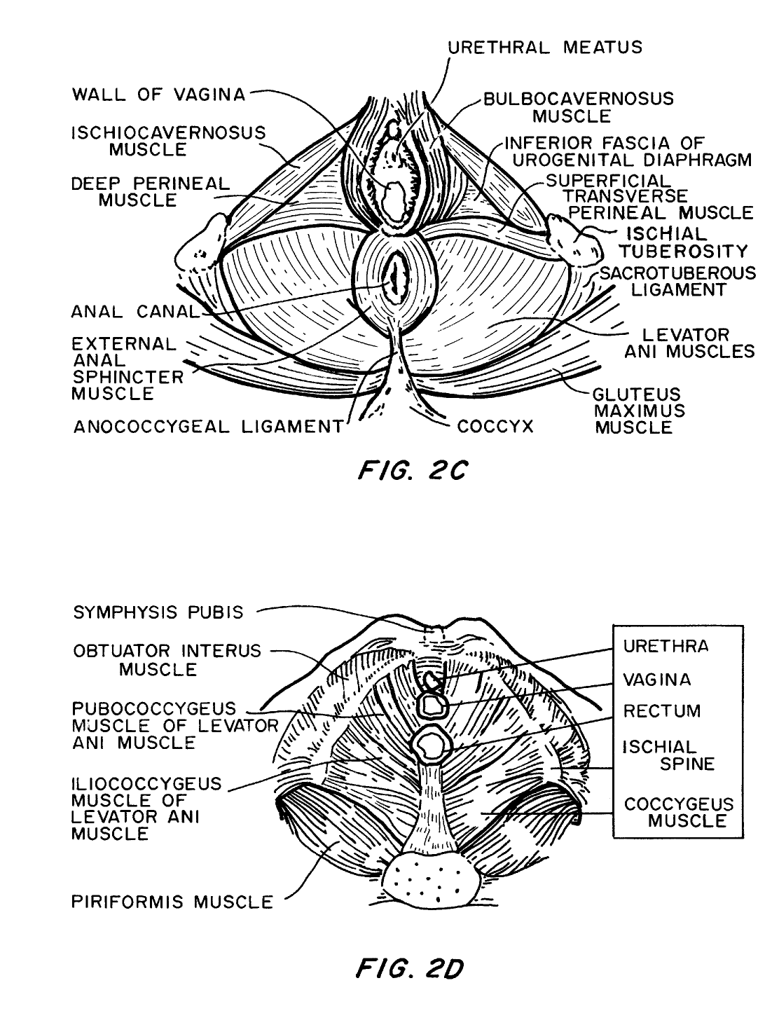

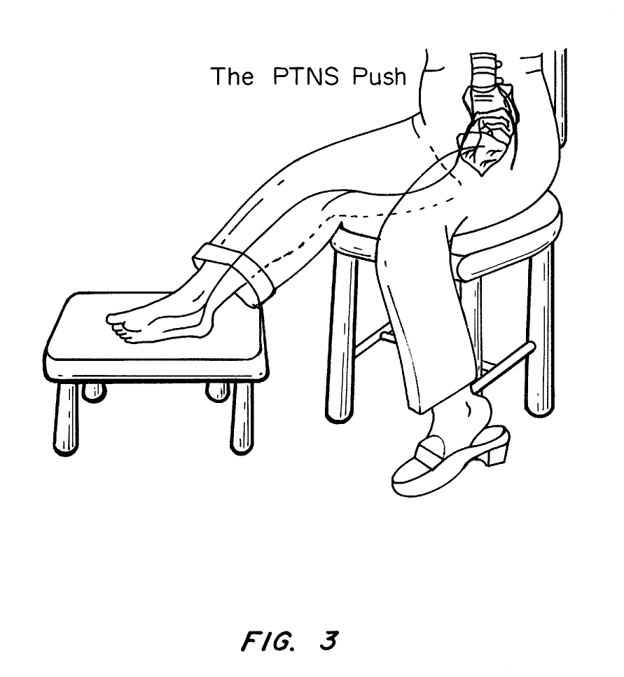

As shown in FIGS. 2A-2D, the pelvic floor is a series of muscles that form a sling across the opening of the pelvis. These 18 muscles and tendons with surrounding tissue are responsible for keeping all of the pelvic organs (bladder, uterus and rectum) in place and functioning properly. Pelvic floor-related disorders include, but are not limited to, urge incontinence, stress incontinence, pelvic organ prolapse, fecal incontinence, pelvic pain, and severe constipation.

In 1965, the gate control theory, Kobashi, Govier, Ame. J. Obst. Gyn. 169(6):2242-2243 (2003) was proposed for the mechanism of action of neuromodulation by basic scientists and anesthesiologists utilizing neuromodulation for the control of pain. This theory demonstrates that the C-fibers, or small fibers, excite a wide dynamic range of neurons. The beta fibers, or large fibers, also can excite these neurons but they may inhibit the firing of the neurons and close the gate. It is important to understand that the effect of the substantia gelatinosa leads to increased activity of the small fibers which essentially reverses any quieting that has occurred through previous neuromodulation; therefore, overstimulation of the neurons can occur (i.e. sacral implants).

In more modern terminology, neuromodulation can communicate excitation or inhibition. Neuromodulation will dictate how much and when this will occur, that is constant firing versus bursts of energy. The neuronal signals vary in time, space, intensity and character. The character of the signal can alter properties of other neurons as signals, which are referred to as crosstalk. The character of the signal can be strengthening of the synaptic response, or alteration in the voltage-dependence of synaptic potentials. It is conditional on the activity of the cells and there is there is no effect unless the synapses are already activated.

The pelvic floor can be rehabilitated by teaching a patient to control muscles of the pelvic floor, in combination with neuromodulation of the pudental and percutaneous neuromodulation of the tibial nerve (PTNS). Timing of administration of each procedure is variable. In the preferred embodiment, each of these steps takes place within about 24 hours, typically within about 12 hours, more typically within 8 hours. An exemplary treatment regime for stress urinary incontinence includes eight treatments of biofeedback, pelvic floor exercises, and 100 Hz electrical stimulation or neuromodulation of the pudental nerve twice per week, followed by 8 PTNS treatments during the same clinic visit, twice per week. The frequency and interval can range from at least once a week, to once every three days, two days, or once a day. The procedures are preferably done on the same day, but could be done on different days.

### I. Pelvic Floor Rehabilitation and Neuromodulation

A. Pelvic Floor Exercises with Biofeedback

Behavioral modification includes: 1) facilitating bladder emptying by having the patient sit down and relax, especially the pelvic floor muscles, 2) double voiding, 3) increasing the voiding interval in overactive bladders and urge incontinence which is usually done for 15 minutes each week, 4) timed voiding in stress urinary incontinence and large bladders, so as to not overfill the bladder and 5) urgency control with attention tasks/activity deep breathing exercises and affirmations. Pelvic floor muscle contractions are also very helpful in urgency control.

Biofeedback can be used to teach the patient to control muscles of the pelvic floor. For example, the Evadri® Bladder Control System from Hollister is an office-based treatment unit that provides a safe, well-tolerated therapy for bladder control problems by helping to retrain the pelvic muscles that are responsible for proper bladder function. The pelvic floor muscles are retrained during a series of six to eight treatment sessions, each lasting approximately 30 to 45 minutes. At all office-based treatment session, a small sensor is inserted into the vagina. The patient is then asked to do an exercise called a “Kegel,” or contract the pelvic floor muscle as if trying to stop urinary flow. The sensor evaluates muscle strength and allows the muscle contraction to be seen on a screen so that the patient and nurse (or doctor) can determine if the patient is using the correct muscles. Biofeedback, in which patients watch a video monitor of the muscles contracting as they exercise, can help patients identify the pelvic muscles so that they can practice on their own. The Evadri® System combines biofeedback with electric signaling designed to reactivate pelvic muscles that may have lost tone due to inactivity.

Small patch electrode sensors may be placed across the abdominal muscles. Monitoring the activity of these abdominal muscles helps to isolate the pelvic muscles that are needed for good bladder control. In the next phase of the treatment, painless electrical signals (i.e., 100 Hz) are sent to the weakened pelvic muscles, causing them to contract effectively. These contractions help retrain the muscles and decrease the symptoms of urgency/frequency or overactive bladder (i.e., 10 Hz), The key to success is correct identification, isolation and contraction of the pelvic muscles followed by a muscle relaxation phase.

This is a particularly important point in management of stress incontinence. Patients are 80%-90% better at the completion of therapy, at three months later have no leakage whatsoever and are completely cured because after three months more of exercises, the pelvic floor muscles begin to reach their peak strength and are much stronger.

B. Neuromodulation of the Pudental and Hypogastric Nerves

As commonly understood, the hypogastric nerve is either of the two nerve trunks designated right and left that lead from the superior hypogastric plexus into the pelvis to join the inferior hypogastric plexuses. The pudental nerve is a nerve in the pelvic region that innervates the external genitalia of both sexes, as well as sphincters for the bladder and the rectum. It is also the direct route to the hypogastric nerve.

Neuromodulation of the pudental and hypogastric nerves are also used in pelvic floor rehabilitation. A vaginal or rectal probe for administration of an electrical current is applied to the relevant nerves.

In one embodiment for the treatment of urge urinary incontinence, six twice per week treatments of 100 Hz electrical stimulation or neuromodulation of the pudental nerve is followed by six twice weekly treatments of 10 Hz electrical stimulation or neuromodulation of the hypogastric nerve. A vaginal probe is inserted into the vagina, and the pulse amplitude turned up from 0 in 5 second intervals, until the patient can feel a comfortable tingling at 10 Hz or sustained contraction of the pelvic floor muscles at 100 Hz. The treatment duration used in the clinical studies described in the examples was 30 minutes but can vary from about 15 minutes to about 60 minutes. Each of these treatment sessions can then be followed during the same clinic visit with a PTNS therapy (approximately 30 minutes).

C. Percutaneous or Transcutaneous Tibial Nerve Neuromodulation

As commonly understood, the tibial nerve is a branch of the sciatic nerve. The sciatic nerve (also known as the ischiatic nerve) is a large nerve that runs down the lower limb. It is the longest single nerve in the body.

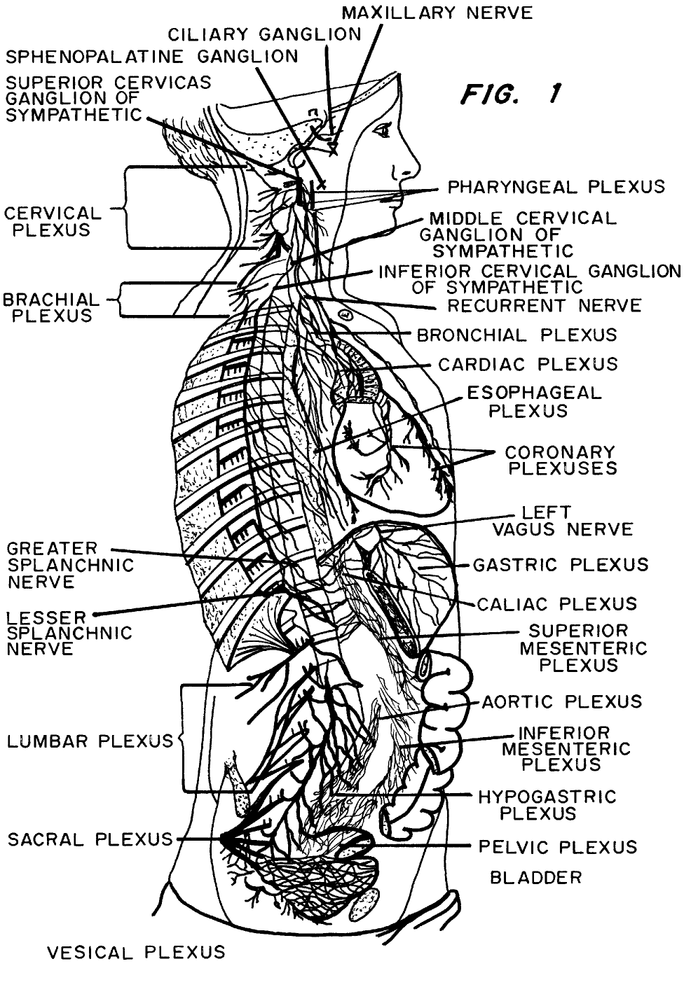

Percutaneous tibial nerve neuromodulation, a therapy that has been approved by the FDA for urge incontinence and overactive bladder, utilizes an accupuncture needle, which is inserted just superior to the medial ankle, to directly stimulate the tibial nerve (3 to 4 cm length), as shown in FIGS. 1 and 3. Similar results can be obtained using transcutaneous neuromodulation using a micro-current pad stimulater, microcurrent probe stimulater, or an interferential device with six preset treatment options to transmit the electric current to the tibial nerve (neuromodulation quiets/relaxes the bladder through parasympathetic and sympathetic pathways by stimulating the sacral plexus, and also Onuf's nucleus, strengthening the urethra). These modes of action have led to its approval by the FDA for urge incontinence and overactive bladders.

It has been discovered that a significant combined effect occurs with biofeedback, pelvic floor exercises, electrical stimulation (10 Hz) to reduce overall bladder muscle (detrusor) activity through the muscarinic cholinergic pathway via the hypogastric nerve, neuromodulation of the pudental nerve (100 Hz) to strengthen the pelvic floor and neuromodulation of the tibial nerve in strengthening the pelvic floor, and quieting the bladder through the sacral complex of nerves.

Tibial nerve neuromodulation leads to neuromodulation of the sacral neurological complex of nerves with a lot of crosstalk affecting the parasympathetic pathways, and the sympathetic pathways. It also strengthens the pelvic floor muscles and urethra while quieting the bladder. PTNS can also be effective for neurogenic bladders. PTNS also neuromodulates Onuf's nucleus, leading to increased levels of norepinephrine and serotonin, which strengthen the urethra and urethral sphincter (sacral implants do not), which leads to strengthening of the urethra and quieting of the bladder even more (via the parasympathetic ganglion).

In one embodiment, percutaneous tibial nerve neuromodulation is administered the same day, typically immediately after the pelvic floor rehabilitation which includes both pelvic floor exercise and pudenal and hypogastric nerve stimulation, but may be administered on different days, or with intervals of 24, 48, 72 or even a week between treatments. The treatment lasts for 15 to about 45 minutes, typically about 30 minutes, for the entire treatment regimes, such as 12 weeks.

The electrical stimulation is applied via a needle, acupuncture needle patch, or skin electrodes. Typically between 0.1 and 50 mA is applied, more preferably 0.25 mA to 9 mA at 9 Hz. In one embodiment, the Urgent PC device can be used. This device is a combination of electrode and generator components including a 34 gauge needle 4 cm in length, surface electrode, lead wires and a hand held electrical generator. The low voltage stimulator has an adjustable pulse intensity of 0 to 9 mA, a fixed pulse width of 200 microseconds and a frequency of 20 Hz. The needle electrode, placed percutaneously above the ankle delivers an electrical impulse produced by the device. This impulse travels via the tibial nerve to the sacral plexus and to Onuf's nucleus. The amplitude of the neuromodulator is then increased from a setting of zero milliamps to 9 milliamps until the patient has a flexion of the great toe. Some patients will actually feel the stimulation up the leg, which is evidence of a successful treatment. In an exemplary regime, patients are treated twice a week ×12 for 30 minutes, at each session.

### II. Disorders to be Treated

A. Urinary Incontinence

The disclosed methods can be used to treat urinary incontinence. Any pathophysiology, such as pelvic muscle attenuation or loss as a result of obstetrical injuries, neurological injury, congenital collagen diseases (hereditary), that affect the pelvic floor musculature and support the bladder itself can lead to urinary incontinence. One of the simplest examples is when a patient has a weakened urethra and coughs; the bladder pressure goes up and is greater than the urethral pressure and the patient will have stress urinary incontinence. There are different terminologies for the various types of types of urinary incontinence. However the most common types are stress incontinence, urge incontinence where the bladder muscle contracts at inappropriate times, (detrusor instability-DI), overflow incontinence, which in women, is usually associated with pelvic organ prolapse (POP) and can be clearly identified and separated from DI with urodynamics, and finally intrinsic sphincter deficiency (ISD), where the closing of the urethral has become ineffective.

One embodiment provides a method for treating urinary urge incontinence including teaching the patient to control muscles of the pelvic floor using biofeedback and electrically stimulating the pudental (100 Hz) and hypogastric (10 Hz) nerves to cause muscles of the pelvic floor to contract, and the bladder muscle (detrusor) to relax. The treatment also includes electrically stimulating the tibial nerve, as well (PTNS), which quiets the bladder and allows it to fill without bladder muscle contraction interruption, which can also cause incontinence. Typically, female patients are treated on a twice weekly basis with six traditional pelvic floor rehabilitations with biofeedback and electrical stimulation at 100 Hz and then an additional six stimulations at 10 Hz using the Hollister Evadri® Bladder Control System. Typically the treatments are alternated with 3 10 Hz, 3 100 Hz, 3 10 Hz, and finally 3 100 Hz sessions. The treatment duration is about 15 to about 60 minutes, typically about 30 minutes. Immediately following the pelvic floor rehabilitation, the patient is treated with tibial nerve neuromodulation for about 15 to 60 minutes, typically about 30 minutes. Tibial nerve neuromodulation can be administered using the commercially available Urgent PC device (Uroplsty Inc.).

For male urinary incontinence, an exemplary treatment regime includes trans-anal administration to the whole pelvis of 100 Hz for four total treatments twice per week followed by neuromodulation of the tibial nerve (PTNS). If the male patient has urge incontinence, then he would receive an additional six trans-anal treatments to the whole pelvis at 1OHz, followed by six PINS sessions. If he has ISD, then a total of 18 PTNS sessions would be administered to strengthen the urethra and urethral sphincter. The neurological pathways are the same in men. In general for both men and women, 10 Hz is only used for urge incontinence, and no matter what the type of incontinence, all patients have the pelvic floor musculature strengthened (100 Hz).

B. Pelvic Organ Prolapse

The disclosed methods can be used to non-surgically treat pelvic organ prolapse. Female patients can be treated with 10 to 15, typically about 12 treatments of 100 Hz electrical stimulations (neuromodulation of the pudental nerve) or any sufficient amount of electricity effective to contract the muscles of the pelvic floor and/or simulate the pudental nerve. All treatments are given twice per week, which has been found to be more effective. The neuromodulation of the pudental nerve is followed by the percutaneous tibial nerve neuromodulation. By rehabilitating the pelvic floor to strengthen the muscles of the pelvic floor, pelvic organ prolapse can be treated with a 97% success rage in Stage 1 patients and an 89% cure rate in Stage 2 patients. As used herein, “success” is rising one fall stage and having no more symptoms. However, prolapse patients may not see anatomical results for two to three months.

C. Fecal Incontinence

Fecal incontinence can be caused by at least three different problems: a weak external sphincter; a weak puborectalis muscle; or failure to recognize the descending bolus of stool. Most commonly, the fecally incontinent patients will have multiple etiologies. A strong pelvic floor is also needed to remain fecally continent. One embodiment provides a method for treating fecal incontinence including applying 100 Hz to the external sphincter (trans-anally six times), 100 Hz treatments in the vagina over the puborectalis muscle six times. Most typically about eight treatments can be administered twice per week. For male patients, the method includes administering 100 Hz to the whole pelvic musculature, for four treatments and then the same treatment to the external sphincter as the female patient (twice per week). As always, all treatments are followed with a PTNS session. Lastly, biofeedback can be used in the rectum with decreasing volumes of air in a balloon, placed into the rectum. Initially, the balloon is filled with air, until the patient can sense the presence of the air filled balloon in the rectum. Each week 10 cc of air is removed from the balloon until the patient regains the ability to recognize even small quantities of stool. The balloon is removed after each biofeedback session, each week.

D. Pelvic Pain

Pelvic pain is typically caused by a weak pelvic floor which then leads to one or both levator muscles going into spasm. This levator spasm is easily detected on pelvic exam, as this area will be extremely tender. One embodiment provides a method for treating pelvic pain including identifying the muscle in spasm and applying 4 to 8 treatments of 10 Hz to the muscle in spasm (angling the probe over the levator in question). It is critical to strengthen the pelvic floor in all of these patients, otherwise, they will relapse very quickly. All treatments are given twice a week and followed by a PINS session. Levator syndrome, as described here, can occur in men as well.

E. Constipation with Fecal Impaction

Constipation with fecal impaction can also be treated using the disclosed methods. An EMG (electromyography) is obtained of both the internal and external anal sphincters to confirm spasm of the internal sphincter and weakness of the external sphincter. In one embodiment, 4 to 8 treatments of 10 Hz are applied to the internal sphincter to stop the spasm. Then 4 to 8 treatments at 100 Hz are applied to the external sphincter (as in fecal incontinence) to strengthen it back to normal. Again the pelvic floor musculature is strengthened in all patients, and every treatment is followed with a PTNS session, except for the internal sphincter treatments. All treatments are given twice per week.

F. Postradical Prostatectomy Incontinence

Incontinence due to prostate surgery can also be treated using the disclosed methods depending on the nature of injury. For incontinence due to damage to the urethral sphincter (most common injury), four treatments of 100 Hz to the whole pelvic musculature is coupled with about 18 treatments of percutaneous tibial nerve neuromodulation (PTNS); (two to three times per week)

Incontinence due to bladder based injury (urge incontinence) can be treated with four to eight treatments of 10 Hz stimulation to the whole pelvis, and then four additional 100 Hz whole pelvic treatments to strengthen the pelvic floor musculature. The method includes about 12 treatments of percutaneous tibial nerve neuromodulation (PINS). All treatments are given twice per week

Incontinence due to pudental nerve injury can be treated with six treatments of 100 Hz delivered trans-anally along with six to twelve treatments of percutaneous tibial nerve neuromodulation (PTNS). All treatments are twice per week.

R. Intrinsic Sphincter Deficiency

Intrinsic sphincter deficiency requires the full strengthening of the pelvic floor as noted above for all patients. However, in addition 18 PTNS sessions are required to strengthen the urethral sphincter and the urethra. Treatments are given twice per week during the PFR, then three times per week after that period of time. If the patient has a continuous drip, the patient will start treatment with a urethral bulking agent and proceed as described above. To date, no patient has required a second injection with this protocol (collagen injections are usually given about every 6 months). This is a major advantage to this approach.

Those skilled in the art will recognize, or be able to ascertain using no more than routine experimentation, many equivalents to the specific embodiments of the invention described herein. Such equivalents are intended to be encompassed by the following claims.

### EXAMPLE 1

### Treatment of Urge Urinary Incontinence

A physician initially saw all the patients for a complete history and physical examination along with a routine urinalysis. A 36-question database was utilized, with the physician asking the questions face to face with the patient. All patients were asked the question, “Did the urge incontinence significantly interfere with your life”. The patient utilized bladder diaries at the beginning, middle, and end of the treatment. If the patient had previous surgery for incontinence, significant pelvic organ prolapse, significant insensible loss, a neurological injury, or any ambiguities in diagnosis of their incontinence, then urodynamic testing was administered. Patients with non-compliant bladders, neurogenic bladders, and significant intrinsic sphincter deficiency (ISD) were excluded from the clinical trial.

Treatment

Constipation was felt to be a significant contributing factor to the problem of urge incontinence. The constipation was always treated with increased fluids and exercise, and a high fiber diet. Senna and colace was routinely prescribed. Polyparaflex was added for a slow colonic transit time as evidenced by pellet-shaped stool, while Lactulose was given for fecal impaction. Patients with urge incontinence were advised to follow strict dietary modifications, which included the avoidance of caffeine in all forms. Patients performed self assessment testing to determine if acidic foods were bladder irritants. If so, these foods were eliminated from their diet. Nurses coached the patients in behavioral modification, in particular learning to lengthen the bladder voiding interval. The nurses also reinforced dietary restrictions and the importance of twice a day pelvic floor exercises.

Patients were treated on a twice per week basis with six electrical stimulations at 100 Hz (pudental nerve neuromodulation), and 6 electrical stimulations at 10 Hz (hypogastric nerve neuromodulation) along with biofeedback and pelvic floor exercises. A vaginal probe was inserted into the vagina and the pulse amplitude turned up from zero, in 5-second intervals, until the patient perceived contractions of the pelvic floor muscles at 100 Hz or buzzing of the muscles at 10 Hz. The pulse width was 1, type balanced and biphasic, without dc component. This was felt to also be excellent biofeedback for their exercises as well. The treatment duration was 30 minutes. Exercises were performed twice daily in the standing, sitting and laying down positions, Patients held the contraction for 5 seconds, then rested for 10 seconds, for 10 repetitions in each position.

The same day, after the PFR, the patients were treated with tibial nerve neuromodulation (PTNS) for 30 minutes (12 treatments). The Urgent PC device is a combination of electrode and generator components including a small 34-gauge needle 4 cm in length, a surface electrode, lead wires and a hand held electrical generator. The low voltage (9 volts) stimulator has an adjustable pulse intensity of zero to 9 mA, a fixed pulse width of 200 microseconds and a frequency of 20 Hz. The needle electrode, placed percutaneously above the ankle delivers an electrical impulse produced by the device. This impulse travels up to the sacral plexus and to Onuf's nucleus via the tibial nerve.

The Uroplasty acupuncture needle was placed in the soft tissue medial and above the ankle to stimulate the tibial nerve directly; pecutaneous tibial nerve stimulation (PTNS). The amplitude of the neuromodulator was then increased from a setting of 0 mA up to 9 mA until the patient had a flexor contraction of the great toe. Some patients would actually feel the stimulation up the leg, which was felt to be adequate documentation of a successful treatment. The patients were treated weekly ×12 for 30 minutes, twice per week.

Vaginal estrogen cream, or pomegranate oil (an ongoing study for patients' with breast cancer) was utilized in all patients receiving therapy, to optimize bladder and pelvic floor muscle function and to avoid any discomfort during pelvic floor rehabilitation (electrical stimulation and biofeedback with pelvic floor exercises). The pelvic floor rehabilitation therapies were done by three RN-BSNs and evaluated by the physician. The physician or nurse practitioner did all of the percutaneous tibial nerve neuromodulations (PINS). The physician interpreted the results of urodynamics testing.

During therapy the physician and nurses monitored patients closely, with frequent communication between each other as to the patient's progress. Patients were followed at three-month intervals for the first year, then every six months thereafter.

Results

This was a prospective trial including 256 patients with urge urinary incontinence who were treated at the Southern Arizona Urogynecology Center in Tucson, Ariz. The patients were referred by primary care physicians, obstetrician gynecologists, urologists, neurologists and self-referrals. The age ranged from 40-87 years and the median age was 62 years. The duration of incontinence varied from four months to greater than 20 years with the median duration being three years. The mean number of accidents per day was 2. In the initial interview, all of these patients answered yes to the question, “Does the stress incontinence significantly interfere with your life?” 7% of the patients (18 patients) were initially eliminated by urodynamics as they did not meet the criteria, noted above for urge incontinence for this prospective trial. Only 3% of all the patients had any pelvic floor strength noted on the initial physical exam.

The criterion for successful treatment was no incontinent episodes, and an OAB-V8 of less than 8 at the three month clinic follow-up visit.

The results in these 238 patients with urge incontinence revealed a 93% success (dry) rate. The remaining 7% of patients had a mean improvement in their incontinence of 84%. The minimal improvement in this trial was 70%. At the end of the trial, all the patients answered negatively to the question, “Does the incontinence significantly interfere with your quality of your life”. The mean OAB-V8 at the 3 month clinic visit was 3.2.

There were four patients who were initially diagnosed as dry after their initial treatment and returned with recurrence of their incontinence, at the 4 month follow-up visit. They were not doing their exercises as instructed, and were back on small amounts of caffeine and acidic foods. After reeducation and a visit at six months, they did not have any incontinence, and are still continent with a mean follow-up of 16 months.

There was no morbidity or complications in any of these 256 patients. The worst side effect was one patient having tingling in her right leg for 1 day. Median follow is now 19 months in the 256 patients.

### EXAMPLE 2

### Treatment of Patients for Urinary Incontinence

208 consecutive female patients with stress incontinence were treated sequentially with the two neuromodulation techniques (PFR and PTNS). Their ages ranged from 38-91 with the median age being 66. The median duration of incontinence was five years; mean number of stress incontinent episodes was 2.5 per day. The stress incontinence was treated with pelvic floor rehabilitation (PFR): eight twice weekly treatments of biofeedback, pelvic floor exercises, and 100 Hz electrical stimulation or neuromodulation of the pudental nerve, followed on the same day with 8 PTNS sessions (twice weekly).

Materials and Methods

A physician completed a history and physical examination on all patients, and a routine urinalysis was done. The physician also administered a questionnaire to the patient, collecting data on 41 relevant points. One question was whether urinary incontinence symptoms significantly interfered with the patient's life. This question was later repeated at the 3 month clinic follow-up visit. The OAB-V8 questionnaire for overactive bladders was utilized in all patients at the first and all subsequent visits. The patient utilized bladder diaries at the beginning, middle, and end of the treatment. If there was ever a doubt as to the diagnosis or if the patient had previous surgery, significant pelvic organ prolapse, significant insensible loss, or neurological injury then urodynamic testing was completed. Final treatment planning occurred after urodynamic interpretation. Patients with urge incontinence, significant intrinsic sphincter deficiency, or a non-compliant bladders were excluded from the study. They represented only four, or 5%, of the patients studied.

208 consecutive female patients with stress incontinence were treated simultaneously with the PFR and PINS techniques. Their ages ranged from 38-91 with the median age being 66. The median duration of incontinence was five years; mean number of urge incontinent episodes was 2.5 per day. The stress incontinence was treated with pelvic floor rehabilitation: eight twice weekly treatments of biofeedback, pelvic floor exercises, and 100 Hz electrical stimulation or neuromodulation of the pudental nerve, followed by eight twice weekly treatments of PTNS. The vaginal probe was inserted into the vagina, and the pulse amplitude turned up from 0 in 5 second intervals, until the patient could feel a comfortable sustained contraction of the pelvic floor muscles at 100 Hz. The pulse width was 1. The pulse type was balanced, biphasic, without a dc component, The treatment duration was 30 minutes. This electrical stimulation is a neuromodulation of the pudental nerve. The same day, immediately after the pelvic floor rehabilitation, all patients received percutaneous tibial nerve modulation (PTNS) for 30 minutes for the entire 4 weeks. The Urgent PC device is a combination of electrode and generator components including a 34 gauge needle 4 cm in length, surface electrode, lead wires and a hand held electrical generator. The low voltage (9 volts) stimulator has an adjustable pulse intensity of 0 to 9 mA, a fixed pulse width of 200 microseconds and a frequency of 20 Hz. The needle electrode, placed percutaneously above the ankle delivers an electrical impulse produced by the device. This impulse travels via the tibial nerve to the sacral plexus and to Onuf's nucleus. The amplitude of the neuromodulator was then increased from a setting of zero milliamps to 9 milliamps until the patient had a flexion of the great toe. Some patients would actually feel the stimulation up the leg, which was also documentation of a successful treatment. The patients were treated twice per week ×8 (4 weeks) for 30 minutes.

Constipation was a significant contributing factor to the problem of stress incontinence in 52% of the patients. It was treated with increased fluids, increased exercise, and a high fiber diet. Senna and colace-two in the morning and at bedtime was routinely prescribed. Miralax was ordered for a slow colonic transit time, as evidenced by pellet-shaped stool. Lactulose was given for fecal impaction.

All patients were treated with the usual dietary modifications recommended for urge incontinence symptoms: the avoidance of caffeine in all forms and acidic foods. Patients tested themselves for the acidic foods to see whether they were bladder irritants or not for them. They stopped the acidic food for 4-7 days, depending on the frequency of intake of that food. If they then improved after testing, they eliminated that item from their diet. Nurses coached the patients in behavioral modification, particularly trying to avoid overfilling of their bladders (voiding schedules) and adhere to dietary restrictions, Vaginal estrogen cream or pomegranate oil (an ongoing study for patient's with breast cancer) was utilized in all patients receiving therapy. This was done to optimize bladder function and pelvic floor function and to avoid any discomfort during pelvic floor rehabilitation. The pelvic floor rehabilitation therapies were done by three RN-BSNs and evaluated by the physician. The physician or nurse practitioner performed all of the percutaneous tibial nerve stimulations and the physician interpreted the urodynamic results. During therapy the physician and nurses monitored patients closely, and communicated with each other as to each patient's progress.

Results

This prospective study included 208 female patients with urinary urge incontinence, who met the study enrollment requirements, and then received treatment at the Southern Arizona Urogynecology Center, Tucson, Ariz. Primary care physicians, obstetrician gynecologists, urologists and neurologists referred the patients; some sought consultation on their own. Ages ranged from 38-91 years, with a median age of 66 years. The duration of incontinence varied from four months to greater than 20 years, with a median duration of five years. The mean number of accidents per day was 2.5.

The criterion for successful treatment, or efficacy was both the absence of incontinent episodes and an OAB V8 score less than 8, indicating no significant overactive bladder symptoms. Of the 208 patients in the study, 91% achieved a totally dry status at the 12 week follow-up visit. The remaining 9% of patients had a median improvement in their incontinence of 81%; no patient improved less than 60%, and all felt that the treatment had significantly improved their quality of life.

Six patients were initially diagnosed as dry, but later returned for their four month follow-up visit with relapse of their incontinence. None were doing their exercises as instructed, and all were consuming small amounts of caffeine and acidic foods. After reeducation, all of these patients were fully continent at a repeat visit four months later and did not have overactive bladders. All are still continent with a mean follow-up of 13 months. No patient has required additional treatment.

There were no infections, bleeding or other serious adverse events in any of these 208 patients. One minor adverse event occurred: following a treatment, one patient had tingling in her pelvis for 2 and ½ days. Median follow is now 20 months in the 208 patients.

Pelvic floor electrical stimulation with 100 Hz directly contracts pelvic floor muscles and indirectly contracts these same muscles through the pudental nerve along with a reflex stimulation providing a wider spread of muscle activation. Treating urge incontinence at 10 Hz with the electrical stimulation operates through reflex mechanisms that neuromodulate the hypogastric efferent nerves, and stimulates the efferent input through the pudental nerve leading to bladder inhibition.

The combination of neuromodulation of the pudental nerve and hypogastric nerves, biofeedback, pelvic floor exercises and neuromodulation of the tibial nerve has a synergistic effect, the result of addressing all three of the neurological pathways to the bladder and strengthening the urethra. The muscarinic cholinergic system is treated through the pudental nerve with electrical stimulation at 10 Hz. The parasympathetic and sympathetic pathways are treated with the neuromodulation of the tibial nerve.

The success rates for percutaneous tibial neuromodulation alone for urge incontinence are 70% improvement and 46% of the patients are cured (Vandonick, et al. Neurourol urodyn. 2004;22:17-23). In another long-term follow-up study, 45% were completely dry; the remaining patients achieved a 75% reduction in their incontinent episodes.

The success rate of 93% using the disclosed treatment regime, with the remaining 7% of patients having a mean 84% improvement for their urge incontinence, documents the significant and unexpected effect of the combination of these two treatment modalities.

Modifications and variations of the methods described herein will be apparent to those skilled in the art from the foregoing detailed description and are intended to come within the scope of the appended claims. All references cited herein are specifically incorporated by reference.

I claim:

1. A method for rehabilitating the pelvic floor of a patient with incontinence, wherein the patient is performing pelvic floor exercises comprising:
electrically stimulating with a neuromodulator the pudental and hypogastric nerves; and
electrically stimulating with a neuromodulator the tibial nerve until the patient has sufficient stimulation to induce flexion of the great toe.

2. The method of claim 1 further comprising providing biofeedback by electrical stimulation of the pelvic floor muscles when the patient is performing pelvic floor exercises.

3. The method of claim 1, comprising electrically stimulating the tibial nerve by placement of an electrode on the tibial nerve above the anlcle to deliver an electrical impulse to the sacral plexus and to Onuf's nucleus.

4. The method of claim 1, wherein between 5 and 100 Hz is used to electrically stimulate the pudental and hypgastric nerves.

5. The method of claim 1, wherein the pudental and hypogastric nerves are electrically stimulated for about 15 minutes to 1 hour.

6. The method of claim 1 wherein the amplitude of the neuromodulator applying stimulation to the pudental and hypogastric nerves is increased from a setting of zero milliamps to 9 milliamps until the patient had a flexion of the great toe.

7. The method of claim 1 comprising applying between 0.1 and 15 mA electrical stimulation to the tibial nerve.

8. The method of claim 1 wherein the pelvic exercises and neuromodulation are performed on the same day.

9. The method of claim 8 wherein neuromodulation of the pudental and hypogastric nerves and the tibial nerve are performed on the same day.

10. The method of claim 1 wherein the patient is treated with electrical stimulation biofeedback by administration of 100 Hz intravaginally or trans-anally to the whole pelvic floor musculature until there is a sustained contraction of the pelvic floor muscles.

11. The method of claim 1 comprising 10 Hz electrical stimulation of the hypogastric nerve by insertion of a vaginal probe into the vagina, or anal probe into the rectum, with the pulse amplitude turned up from 0 in 5 second intervals, until the patient can feel a comfortable tingling, wherein the pulse width is 1, and the pulse type is balanced, biphasic, and without a dc component, for a treatment duration of 30 minutes.

12. The method of claim 1 wherein percutaneous tibial nerve modulation is administered for 30 minutes

13. The method of claim 14 wherein the tibial nerve modulation is administered with a low voltage (9 volts) stimulator having an adjustable pulse intensity of 0 to 9 mA, a fixed pulse width of 200 microseconds and a frequency of 20 Hz, wherein the electrode is placed above the ankle to deliver an electrical impulse via the tibial nerve to the sacral plexus and to Onuf's nucleus, and wherein the amplitude of the neuromodulator is then increased from a setting of zero milliamps to 9 milliamps until the patient had a flexion of the great toe.

14. The method of claim 1 wherein the patient is treated with twice weekly treatments of biofeedback, neuromodulation of the pudential and hypogastric nerve, followed by neuromodulation of the tibial nerve.

15. The method of claim 1 wherein the pelvic floor-related disorder is selected from the group consisting of urge incontinence and stress incontinence.

16. The method of claim 1 wherein the patient is treated for a condition selected from the group consisting of pelvic organ prolapse, fecal incontinence, pelvic pain, constipation with fecal impaction, post-radical prostatectomy incontinence, and intrinsic sphincter deficiency.

17. The method of claim 1, wherein the tibial nerve stimulation is performed trans-cutaneously.

18. The method of claim 17 using a microcurrent pad stimulator, a microcurrent probe stimulator, an interferential device using a form of electrostimulation that utilized four electrodes surrounding the part of the body to be treated, with each set of two electrodes at a frequency out-of-phase with the other set; a high-intensity form of interferential current, or a Russian stimulation device.

19. The method of claim 17 using therapeutic microcurrent stimulation at a level several hundred to a thousand times less intense than conventional milliamp electro-stimulators using specific electrodes to treat specific points on the body with microcurrent stimulation and measuring skin impedance to confirm optimal electrical conductivity and placement.

20. The method of claim 1 wherein the patient is a woman.

21. The method of claim 1 wherein the patient is a man.
